(19) United States
(12) Patent Application Publication
(10) Pub. No.: US 2026/0225596 A1
(43) Pub. Date: Aug. 6, 2026

(54) DROWSINESS DETERMINATION DEVICE AND DROWSINESS DETERMINATION METHOD

(71) Applicant: Mitsubishi Electric Corporation, Tokyo (JP)

(72) Inventor: Koki ABE, Tokyo (JP)

(73) Assignee: Mitsubishi Electric Corporation, Tokyo (JP)

(21) Appl. No.: 19/148,808

(22) PCT Filed: Mar. 6, 2023
(86) PCT No.: PCT/JP2023/008206

§ 371 Date: Jul. 17, 2025

(51) Int. Cl. B60W 40/08; B60W 50/00; G06V 20/59; G06V 40/18

(52) U.S. Cl. CPC B60W 40/08; B60W 50/00; G06V 20/597; G06V 40/18; B60W 2040/0827; B60W 2050/0029; B60W 2420/403; B60W 2540/221; B60W 2540/229

(57) ABSTRACT

A drowsiness determination device includes processing circuitry configured to: acquire image data indicating a face image of a driver from a camera that captures a face of the driver; estimate an alertness level indicating a degree of alertness of the driver on a basis of the acquired image data; acquire waveform information indicating an electrocardiographic waveform of the driver from a sensor that detects an electrocardiographic waveform of the driver; calculate a feature amount of a heartbeat of the driver from the electrocardiographic waveform indicated by the acquired waveform information; determine presence or absence of drowsiness of the driver on a basis of the estimated alertness level and the calculated feature amount of the heartbeat.

### TECHNICAL FIELD

The present disclosure relates to a drowsiness determination device and a drowsiness determination method.

### BACKGROUND ART

There is a drowsiness determination device that determines whether or not a driver is drowsy.

As such a drowsiness determination device, for example, Patent Literature 1 discloses a device including a drowsiness sensor.

The drowsiness sensor senses drowsiness of the driver on the basis of heartbeat intervals obtained from the electrocardiographic waveform of the driver.

### CITATION LIST

### Patent Literatures

Patent Literature 1: JP 2019-13737 A

### SUMMARY OF INVENTION

### Technical Problem

The heartbeat intervals are generally longer when the driver is drowsy than when the driver is not drowsy. However, even when the driver is not drowsy, the heartbeat intervals vary depending on the activity status of the autonomic nerve. For example, the driver relaxes more as a certain period elapses after the start of driving than when the driver starts driving the vehicle, so that the activity situation of the autonomic nerve changes, and the heartbeat interval may become longer even if the driver has not become drowsy.

In the device disclosed in Patent Literature 1, the drowsiness sensor senses the drowsiness of the driver only on the basis of the heartbeat intervals. Thus, there is a problem that the drowsiness sensor erroneously detects drowsiness when the driver is not drowsy.

The present disclosure has been made to solve the above problems, and an object of the present disclosure is to obtain a drowsiness determination device that can reduce false detection of drowsiness more than the device disclosed in Patent Literature 1.

### Solution to Problem

A drowsiness determination device according to the present disclosure includes: an image data acquiring unit to acquire image data indicating a face image of a driver from a camera that captures a face of the driver; an alertness level estimating unit to estimate an alertness level indicating a degree of alertness of the driver on the basis of the image data acquired by the image data acquiring unit; a waveform information acquiring unit to acquire waveform information indicating an electrocardiographic waveform of the driver from a sensor that detects an electrocardiographic waveform of the driver; and a heartbeat feature amount calculating unit to calculate a feature amount of a heartbeat of the driver from the electrocardiographic waveform indicated by the waveform information acquired by the waveform information acquiring unit. Further, the drowsiness determination device further includes: a drowsiness presence-absence determination unit to determine presence or absence of drowsiness of the driver on the basis of the alertness level estimated by the alertness level estimating unit and the feature amount of the heartbeat calculated by the heartbeat feature amount calculating unit.

### Advantageous Effects of Invention

According to the present disclosure, it is possible to reduce false detection of drowsiness more than the device disclosed in Patent Literature 1.

### BRIEF DESCRIPTION OF DRAWINGS

FIG. 1 is a configuration diagram illustrating a drowsiness determination device 3 according to a first embodiment.

FIG. 2 is a hardware configuration diagram illustrating hardware of the drowsiness determination device 3 according to the first embodiment.

FIG. 3 is a hardware configuration diagram of a computer in a case where the drowsiness determination device 3 is implemented by software, firmware, or the like.

FIG. 4 is a flowchart illustrating a target detection method that is a processing procedure of the drowsiness determination device 3.

FIG. 5A is an explanatory diagram illustrating a distance L1 between an upper eyelid and a lower eyelid when a driver is not drowsy, and FIG. 5B is an explanatory diagram illustrating a current distance L2 between the upper eyelid and the lower eyelid of the driver.

FIG. 6 is an explanatory diagram illustrating an example of heartbeat intervals of the driver.

FIG. 7 is a configuration diagram illustrating a drowsiness determination device 3 according to a second embodiment.

FIG. 8 is a hardware configuration diagram illustrating hardware of the drowsiness determination device 3 according to the second embodiment.

FIG. 9 is a configuration diagram illustrating a drowsiness determination device 3 according to a third embodiment.

FIG. 10 is a hardware configuration diagram illustrating hardware of the drowsiness determination device 3 according to the third embodiment.

### DESCRIPTION OF EMBODIMENTS

Hereinafter, in order to describe the present disclosure in more detail, modes for carrying out the present disclosure will be described with reference to the accompanying drawings.

### First Embodiment

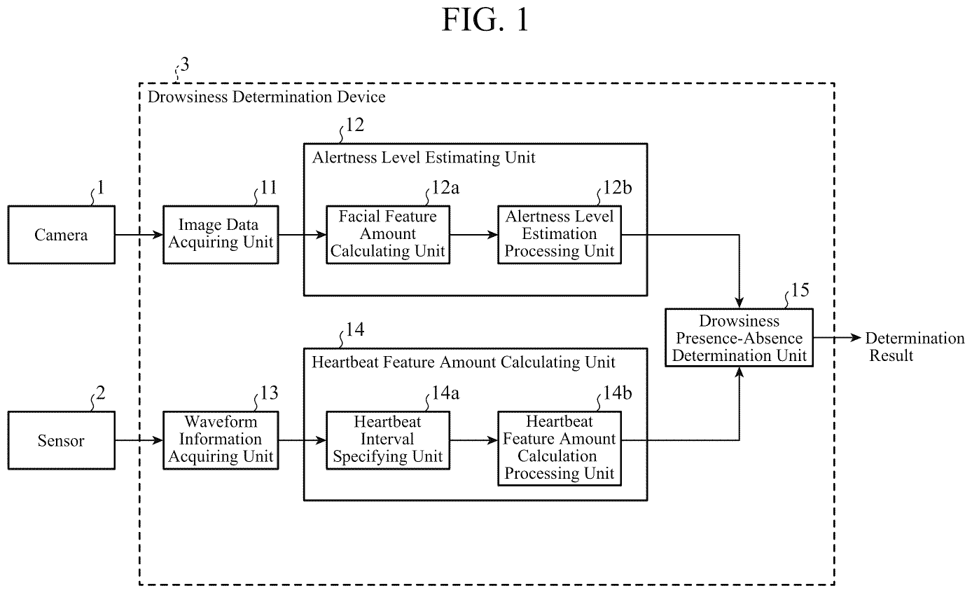

FIG. 1 is a configuration diagram illustrating a drowsiness determination device 3 according to a first embodiment.

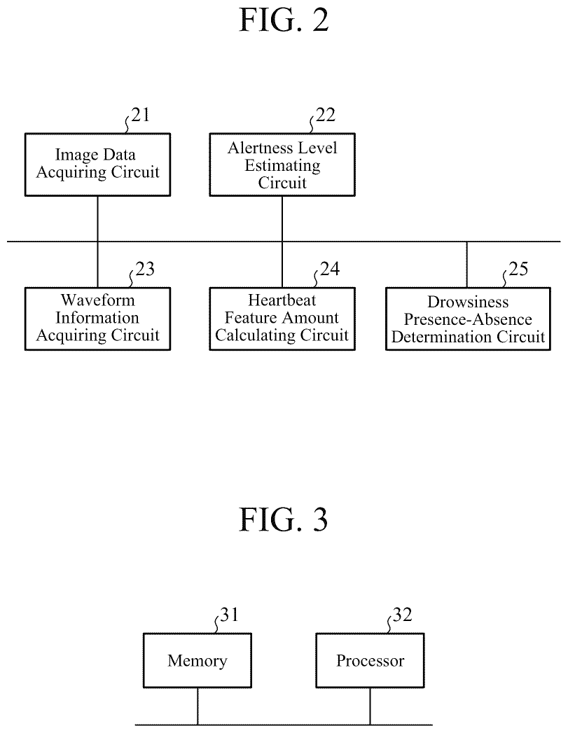

FIG. 2 is a hardware configuration diagram illustrating hardware of the drowsiness determination device 3 according to the first embodiment.

In FIG. 1, a camera 1 is installed, for example, on the instrument panel of a vehicle, the windshield of the vehicle, or the ceiling of the vehicle.

The camera 1 is implemented by, for example, one or more visible light cameras, one or more infrared cameras, or a video camera. In a case where the camera 1 is implemented by an infrared camera, a light source that emits infrared rays for imaging may be provided in an area including the driver's face. The light source is implemented by, for example, a light emitting diode (LED).

The camera 1 captures the face of the driver and outputs image data indicating a face image of the driver to the drowsiness determination device 3.

A sensor 2 is implemented by, for example, an electrocardiographic sensor that detects an electrocardiographic waveform of the driver in a state of being in contact with the driver, or an electrocardiographic sensor that detects an electrocardiographic waveform of the driver in a state of not being in contact with the driver.

The sensor 2 outputs waveform information indicating an electrocardiographic waveform of the driver to the drowsiness determination device 3.

The sensor 2 is not limited to one implemented by an electrocardiographic sensor, and may be implemented by an infrared camera. In a case where the sensor 2 is implemented by an infrared camera, the sensor 2 detects an electrocardiographic waveform of the driver on the basis of luminance of a face surface that changes with a heartbeat of the driver.

The drowsiness determination device 3 includes an image data acquiring unit 11, an alertness level estimating unit 12, a waveform information acquiring unit 13, a heartbeat feature amount calculating unit 14, and a drowsiness presence-absence determination unit 15.

The drowsiness determination device 3 determines whether the driver is drowsy on the basis of the image data output from the camera 1 and the waveform information output from the sensor 2.

The image data acquiring unit 11 is implemented by, for example, an image data acquiring circuit 21 illustrated in FIG. 2.

The image data acquiring unit 11 acquires the image data indicating the face image of the driver from the camera 1.

The image data acquiring unit 11 outputs the image data to the alertness level estimating unit 12.

The alertness level estimating unit 12 is implemented by, for example, an alertness level estimating circuit 22 illustrated in FIG. 2.

The alertness level estimating unit 12 includes a facial feature amount calculating unit 12a and an alertness level estimation processing unit 12b.

The alertness level estimating unit 12 acquires the image data from the image data acquiring unit 11.

The alertness level estimating unit 12 estimates an alertness level indicating the degree of alertness of the driver on the basis of the image data. The alertness level is an apparent alertness level of the driver estimated from the face image of the driver.

The alertness level estimating unit 12 outputs the alertness level of the driver to the drowsiness presence-absence determination unit 15.

The facial feature amount calculating unit 12a acquires image data from the image data acquiring unit 11.

The facial feature amount calculating unit 12a detects the face of the driver from the image data and calculates the feature amount of the face.

The facial feature amount calculating unit 12a calculates a normalization coefficient of the facial feature amount on the basis of a plurality of pieces of image data acquired by the image data acquiring unit 11.

The plurality of pieces of image data is a plurality of pieces of image data at different times of capturing by the camera 1, and includes the latest image data output from the image data acquiring unit 11 and past image data output before the latest image data from the image data acquiring unit 11. For example, when the number of pieces of data of the plurality of pieces of image data is N, the plurality of pieces of image data includes (N−1) pieces of past image data in addition to the latest image data. The N pieces of image data are time-series data, and N is an integer equal to or more than 2.

The facial feature amount calculating unit 12a normalizes the feature amount of the face using the normalization coefficient.

The facial feature amount calculating unit 12a outputs the feature amount of the face after normalization to the alertness level estimation processing unit 12b.

The alertness level estimation processing unit 12b acquires the feature amount of the face after normalization from the facial feature amount calculating unit 12a.

The alertness level estimation processing unit 12b estimates the alertness level of the driver on the basis of the feature amount of the face after normalization.

The alertness level estimation processing unit 12b outputs the alertness level of the driver to the drowsiness presence-absence determination unit 15.

The waveform information acquiring unit 13 is implemented by, for example, a waveform information acquiring circuit 23 illustrated in FIG. 2.

The waveform information acquiring unit 13 acquires, from the sensor 2, waveform information indicating an electrocardiographic waveform of the driver.

The waveform information acquiring unit 13 outputs the waveform information to the heartbeat feature amount calculating unit 14.

The heartbeat feature amount calculating unit 14 is implemented by, for example, a heartbeat feature amount calculating circuit 24 illustrated in FIG. 2.

The heartbeat feature amount calculating unit 14 includes a heartbeat interval specifying unit 14a and a heartbeat feature amount calculation processing unit 14b.

The heartbeat feature amount calculating unit 14 acquires the waveform information from the waveform information acquiring unit 13.

The heartbeat feature amount calculating unit 14 calculates the feature amount of the heartbeat of the driver from the electrocardiographic waveform indicated by the waveform information.

The heartbeat feature amount calculating unit 14 outputs the feature amount of the heartbeat to the drowsiness presence-absence determination unit 15.

The heartbeat interval specifying unit 14a acquires the waveform information from the waveform information acquiring unit 13.

The heartbeat interval specifying unit 14a specifies a heartbeat interval of the driver on the basis of the electrocardiographic waveform indicated by the waveform information.

The heartbeat interval specifying unit 14a outputs the heartbeat interval of the driver to the heartbeat feature amount calculation processing unit 14b.

The heartbeat feature amount calculation processing unit 14b acquires a heartbeat interval of the driver from the heartbeat interval specifying unit 14a.

The heartbeat feature amount calculation processing unit 14b calculates the feature amount of the heartbeat from the heartbeat interval.

The heartbeat feature amount calculation processing unit 14b calculates a normalization coefficient of the feature amount of the heartbeat on the basis of the plurality of pieces of waveform information acquired by the waveform information acquiring unit 13.

The plurality of pieces of waveform information is a plurality of pieces of waveform information at different times of detection by the sensor 2, and includes the latest waveform information output from the waveform information acquiring unit 13 and past waveform information output before the latest waveform information from the waveform information acquiring unit 13. For example, when the number of pieces of data of the plurality of pieces of waveform information is M, the plurality of pieces of waveform information includes (M−1) pieces of past waveform information in addition to the latest waveform information. The M pieces of waveform information are time-series data, and M is an integer equal to or more than 2.

The heartbeat feature amount calculation processing unit 14b normalizes the feature amount of the heartbeat using the normalization coefficient.

The heartbeat feature amount calculation processing unit 14b outputs the feature amount of the heartbeat after normalization to the drowsiness presence-absence determination unit 15.

The drowsiness presence-absence determination unit 15 is implemented by, for example, a drowsiness presence-absence determination circuit 25 illustrated in FIG. 2.

The drowsiness presence-absence determination unit 15 determines the presence or absence of drowsiness of the driver on the basis of the alertness level estimated by the alertness level estimating unit 12 and the feature amount of the heartbeat calculated by the heartbeat feature amount calculating unit 14.

Specifically, the drowsiness presence-absence determination unit 15 acquires the alertness level of the driver from the alertness level estimation processing unit 12b, and acquires the feature amount of the heartbeat after normalization from the heartbeat feature amount calculation processing unit 14b.

Then, the drowsiness presence-absence determination unit 15 determines the presence or absence of drowsiness of the driver on the basis of the alertness level of the driver and the feature amount of the heartbeat after normalization.

In FIG. 1, it is assumed that each of the image data acquiring unit 11, the alertness level estimating unit 12, the waveform information acquiring unit 13, the heartbeat feature amount calculating unit 14, and the drowsiness presence-absence determination unit 15, which are components of the drowsiness determination device 3, is implemented by dedicated hardware as illustrated in FIG. 2. That is, it is assumed that the drowsiness determination device 3 is implemented by the image data acquiring circuit 21, the alertness level estimating circuit 22, the waveform information acquiring circuit 23, the heartbeat feature amount calculating circuit 24, and the drowsiness presence-absence determination circuit 25.

Further, each of the image data acquiring circuit 21, the alertness level estimating circuit 22, the waveform information acquiring circuit 23, the heartbeat feature amount calculating circuit 24, and the drowsiness presence-absence determination circuit 25 corresponds to, for example, a single circuit, a composite circuit, a programmed processor, a parallel-programmed processor, an application specific integrated circuit (ASIC), a field-programmable gate array (FPGA), or a combination thereof.

The components of the drowsiness determination device 3 are not limited to those implemented by dedicated hardware, and the drowsiness determination device 3 may be implemented by software, firmware, or a combination of software and firmware.

The software or firmware is stored in a memory of a computer as a program. The computer means hardware that executes a program, and corresponds to, for example, a central processing unit (CPU), a graphics processing unit (GPU), a central processing unit, a processing unit, an arithmetic unit, a microprocessor, a microcomputer, a processor, or a digital signal processor (DSP).

FIG. 3 is a hardware configuration diagram of a computer in a case where the drowsiness determination device 3 is implemented by software, firmware, or the like.

In a case where the drowsiness determination device 3 is implemented by software, firmware, or the like, a program for causing a computer to execute each processing procedure in the image data acquiring unit 11, the alertness level estimating unit 12, the waveform information acquiring unit 13, the heartbeat feature amount calculating unit 14, and the drowsiness presence-absence determination unit 15 is stored in a memory 31. Then, a processor 32 of the computer executes the program stored in the memory 31.

Further, FIG. 2 illustrates an example in which each of the components of the drowsiness determination device 3 is implemented by dedicated hardware, and FIG. 3 illustrates an example in which the drowsiness determination device 3 is implemented by software, firmware, or the like. However, this is merely an example, and some components in the drowsiness determination device 3 may be implemented by dedicated hardware, and the remaining components may be implemented by software, firmware, or the like.

Next, the operation of the drowsiness determination device 3 illustrated in FIG. 1 will be described.

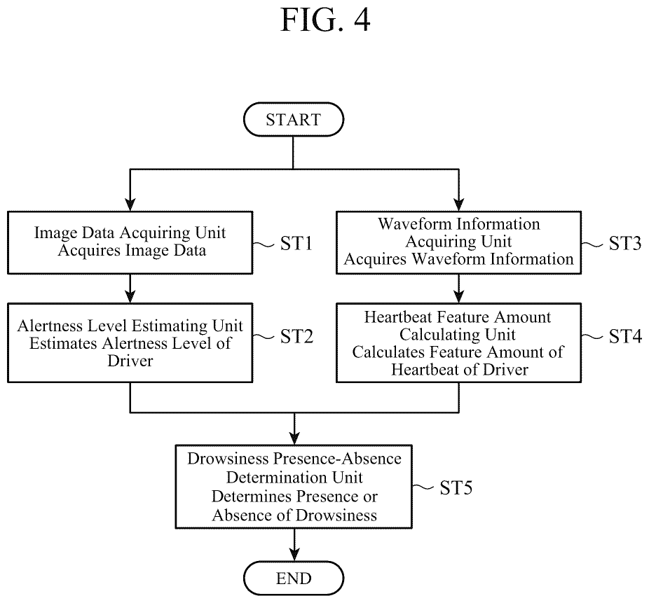

FIG. 4 is a flowchart illustrating a drowsiness determination method that is a processing procedure of the drowsiness determination device 3.

For example, the camera 1 starts capturing the face of a driver from a time point when the driver gets into the vehicle and the driver starts the engine. However, the capturing start time point of the camera 1 is not limited to the time point when the driver starts the engine, and may be, for example, a time point when the driver gets into the vehicle.

The camera 1 outputs image data indicating the face image of the driver to the image data acquiring unit 11 of the drowsiness determination device 3. The face image indicated by the image data may be a moving image or a plurality of still images intermittently captured.

The sensor 2 detects an electrocardiographic waveform of the driver.

The sensor 2 outputs the waveform information indicating the electrocardiographic waveform of the driver to the waveform information acquiring unit 13 of the drowsiness determination device 3.

The image data acquiring unit 11 acquires the image data indicating the face image of the driver from the camera 1 (step ST1 in FIG. 4).

The image data acquiring unit 11 outputs the image data to the alertness level estimating unit 12.

The alertness level estimating unit 12 acquires the image data from the image data acquiring unit 11.

The alertness level estimating unit 12 estimates the alertness level of the driver on the basis of the image data (step ST2 in FIG. 4).

The alertness level estimating unit 12 outputs the alertness level of the driver to the drowsiness presence-absence determination unit 15.

Hereinafter, the estimation processing of the alertness level by the alertness level estimating unit 12 will be specifically described.

The facial feature amount calculating unit 12a acquires the image data from the image data acquiring unit 11.

The facial feature amount calculating unit 12a detects the face of the driver from the face image of the driver indicated by the image data.

Specifically, the facial feature amount calculating unit 12a can detect the face of the driver by using, for example, a Haar-Like detector to which a machine learning algorithm called adaptive boosting (AdaBoost) or an algorithm called cascade is applied. The Haar-Like detector is a detector that handles Haar-Like feature amounts. The Haar-Like feature amount is a feature amount obtained from brightness differences of a plurality of local regions.

The facial feature amount calculating unit 12a calculates the detected facial feature amount.

Specifically, the facial feature amount calculating unit 12a detects a face part from the driver's face. The face part is, for example, an eye of the driver or the mouth of the driver.

Next, the facial feature amount calculating unit 12a calculates, for example, the eye opening degree of the driver's eyes or the opening amount of the driver's mouth.

The eye opening degree of the eye is calculated from, for example, a distance L1 between the upper eyelid and the lower eyelid in the vertical direction of the driver when the driver is not drowsy and a current distance L2 between the upper eyelid and the lower eyelid in the vertical direction of the driver as expressed in the following equation (1). When the driver is seated on the driver's seat and looking forward in the vehicle, the up-down direction of the upper eyelid and the lower eyelid is substantially the same as the vertical direction.

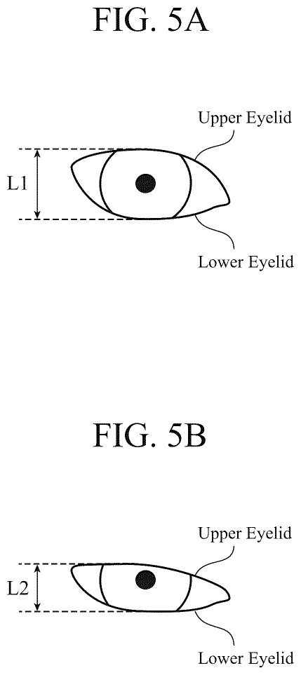

As illustrated in FIG. 5A, the distance L1 is a distance at a position where the distance between the upper eyelid and the lower eyelid is maximized when the driver is not drowsy. The distance L1 is calculated, for example, from an eye of the driver, which is a face part detected until a certain time elapses after the driver gets into the vehicle. The distance L1 is not limited to the distance calculated from the driver's eye detected until a certain time elapses after the driver gets into the vehicle, and may be stored in an internal memory of the facial feature amount calculating unit 12a or may be provided from the outside of the drowsiness determination device 3, for example.

As illustrated in FIG. 5B, the distance L2 is calculated from the driver's eye, which is a face part detected from the face image indicated by the latest image data acquired from the image data acquiring unit 11.

FIG. 5A is an explanatory diagram illustrating the distance L1 between the upper eyelid and the lower eyelid when the driver is not drowsy.

FIG. 5B is an explanatory diagram illustrating the current distance L2 between the upper eyelid and the lower eyelid of the driver.

$$Eye opening degree of eye = ( L 2 / L 1 ) \times 100 \qquad ( 1 )$$

Here, the eye opening degree of the eye is calculated by Expression (1). However, this is merely an example, and the eye opening degree of the eye may be obtained by normalizing the distance L1 in the vertical direction between the upper eyelid and the lower eyelid by a face part such as the nose, the mouth, or an ear.

The opening amount of the mouth is, for example, a distance in the vertical direction between the upper lip and the lower lip at a position where the distance between the upper lip and the lower lip is maximum.

The facial feature amount calculating unit 12a calculates a facial feature amount effective for estimating the alertness level of the driver on the basis of, for example, the eye opening degree of the eyes or the opening amount of the mouth.

Examples of such a feature amount of the face include a ratio of a time during which the eyes are closed within a certain period, the number of blinks in a certain period, and the number of yawns in a certain period.

The ratio of the time during which the eye is closed within a certain period is obtained by dividing the time during which the degree of eye opening becomes 0% within the certain period by the time of the certain period.

The number of blinks in a certain period is obtained from the number of times the eye opening degree reached 0% within the certain period.

Yawning is detected on the basis of a change in the opening amount of the mouth.

The facial feature amount calculating unit 12a normalizes the facial feature amount in order to absorb individual differences in the facial feature amounts.

Specifically, for example, the facial feature amount calculating unit 12a accumulates the facial feature amounts calculated on the basis of each of N pieces of image data including the latest image data and the past image data, and calculates an average value of the accumulated feature amounts or a percentile value of the accumulated feature amounts. Then, the facial feature amount calculating unit 12a sets the calculated average value or the calculated percentile value as a normalization coefficient.

The facial feature amount calculating unit 12a subtracts a normalization coefficient from the facial feature amount calculated on the basis of the latest image data or divides the facial feature amount calculated on the basis of the latest image data by the normalization coefficient, thereby normalizing the facial feature amount.

The facial feature amount calculating unit 12a outputs the feature amount of the face after normalization to the alertness level estimation processing unit 12b.

In the drowsiness determination device 3 illustrated in FIG. 1, the facial feature amount calculating unit 12a accumulates the facial feature amount calculated on the basis of each of the N pieces of image data including the latest image data and the past image data. However, this is merely an example, and the facial feature amount calculating unit 12a may accumulate the facial feature amount calculated on the basis of each of N pieces of image data including only past image data.

Furthermore, the feature amount of the face calculated on the basis of each of the N pieces of image data may be a feature amount of the face calculated during a period from when the driver gets into the vehicle until a certain time elapses.

The alertness level estimation processing unit 12b acquires the feature amount of the face after normalization from the facial feature amount calculating unit 12a.

The alertness level estimation processing unit 12b estimates the alertness level of the driver on the basis of the feature amount of the face after normalization.

Specifically, the alertness level estimation processing unit 12b gives the feature amount of the face after normalization to a learning model using a general algorithm such as random forest or logistic regression, and acquires the alertness level of the driver from the learning model.

At the time of learning, the learning model is given the feature amount of the face after normalization and teacher data indicating the alertness level of the driver, and learns the alertness level of the driver.

When the feature amount of the face after normalization is given from the alertness level estimation processing unit 12b at the time of inference, the learning model outputs the alertness level of the driver to the alertness level estimation processing unit 12b. The alertness level of the driver is, for example, data of 0 to 1.

The alertness level estimation processing unit 12b outputs the alertness level of the driver to the drowsiness presence-absence determination unit 15.

The waveform information acquiring unit 13 acquires, from the sensor 2, the waveform information indicating the electrocardiographic waveform of the driver (step ST3 in FIG. 4).

The waveform information acquiring unit 13 outputs the waveform information to the heartbeat feature amount calculating unit 14.

The heartbeat feature amount calculating unit 14 acquires the waveform information from the waveform information acquiring unit 13.

The heartbeat feature amount calculating unit 14 calculates the feature amount of the heartbeat of the driver from the electrocardiographic waveform indicated by the waveform information (step ST4 in FIG. 4).

The heartbeat feature amount calculating unit 14 outputs the feature amount of the heartbeat to the drowsiness presence-absence determination unit 15.

Hereinafter, calculation processing of the heartbeat feature amount by the heartbeat feature amount calculating unit 14 will be specifically described.

The heartbeat interval specifying unit 14a acquires the waveform information from the waveform information acquiring unit 13.

The heartbeat interval specifying unit 14a specifies a heartbeat interval of the driver on the basis of the electrocardiographic waveform indicated by the waveform information.

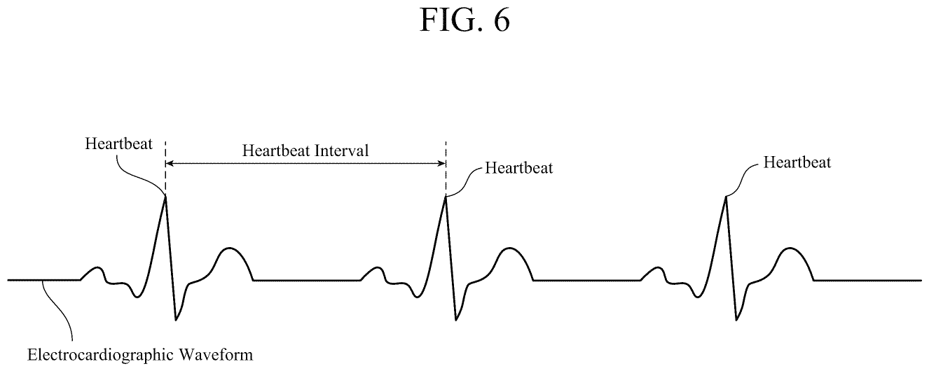

The heartbeat interval is the time between two adjacent heartbeats, as illustrated in FIG. 6.

FIG. 6 is an explanatory diagram illustrating an example of a heartbeat interval of the driver.

The heartbeat interval specifying unit 14a outputs the heartbeat interval of the driver to the heartbeat feature amount calculation processing unit 14b.

The heartbeat feature amount calculation processing unit 14b acquires the heartbeat interval of the driver from the heartbeat interval specifying unit 14a.

The heartbeat feature amount calculation processing unit 14b calculates the feature amount of the heartbeat from the heartbeat interval.

Examples of the feature amount of the heartbeat include the following feature amounts (1) to (6).
(1) Average value of heartbeat intervals of the driver within a certain period
(2) Standard deviation within a certain period of the heartbeat interval of the driver
(3) Average value of driver's heartbeats within a certain period
(4) Square root of the average value within a certain period of squares of difference between two heartbeat intervals adjacent in the time direction
(5) Number of times a difference between two heartbeat intervals adjacent in the time direction becomes larger than a threshold within a certain period

(6) Ratio at which difference between two heartbeat intervals adjacent in time direction becomes larger than threshold within certain period

The heartbeat feature amount calculation processing unit 14b normalizes the feature amount of the heartbeat in order to absorb the individual differences in the feature amounts of the heartbeat.

Specifically, the heartbeat feature amount calculation processing unit 14b acquires, for example, M pieces of waveform information including the latest waveform information and the past waveform information from the waveform information acquiring unit 13 via the heartbeat interval specifying unit 14a.

The heartbeat feature amount calculation processing unit 14b accumulates the feature amount of the heartbeat calculated on the basis of each of the M pieces of waveform information, and calculates an average value of the accumulated feature amounts or a percentile value of the accumulated feature amounts. Then, the heartbeat feature amount calculation processing unit 14b sets the calculated average value or the calculated percentile value as a normalization coefficient.

The heartbeat feature amount calculation processing unit 14b normalizes the feature amount of the heartbeat by subtracting a normalization coefficient from the feature amount of the heartbeat calculated on the basis of the latest waveform information or dividing the feature amount of the heartbeat calculated on the basis of the latest waveform information by the normalization coefficient.

The heartbeat feature amount calculation processing unit 14b outputs the feature amount of the heartbeat after normalization to the drowsiness presence-absence determination unit 15.

In the drowsiness determination device 3 illustrated in FIG. 1, the heartbeat feature amount calculation processing unit 14b accumulates the feature amount of the heartbeat calculated on the basis of each of the M pieces of waveform information including the latest waveform information and the past waveform information. However, this is merely an example, and the heartbeat feature amount calculation processing unit 14b may accumulate the feature amount of the heartbeat calculated on the basis of each of the M pieces of waveform information including only the past waveform information.

Furthermore, the feature amount of the heartbeat calculated on the basis of each of the M pieces of waveform information may be a feature amount of the heartbeat calculated during a period from when the driver gets into the vehicle until a certain time elapses.

The drowsiness presence-absence determination unit 15 acquires the alertness level of the driver from the alertness level estimation processing unit 12b, and acquires the feature amount of the heartbeat after normalization from the heartbeat feature amount calculation processing unit 14b.

The drowsiness presence-absence determination unit 15 determines the presence or absence of drowsiness of the driver on the basis of the alertness level of the driver and the feature amount of the heartbeat after normalization (step ST5 in FIG. 4).

Specifically, for example, the drowsiness presence-absence determination unit 15 gives the alertness level of the driver and the feature amount of the heartbeat after normalization to a learning model using a general algorithm of random forest or logistic regression, and acquires the determination result of the presence or absence of drowsiness from the learning model.

At the time of learning, the learning model is given the alertness level of the driver, the feature amount of the heartbeat after normalization, and the teacher data indicating the presence or absence of drowsiness of the driver, and learns the presence or absence of drowsiness. The teacher data is, for example, data “1” when the driver is not drowsy, and data “0” when the driver is drowsy.

When the alertness level of the driver and the feature amount of the heartbeat after normalization are given from the drowsiness presence-absence determination unit 15 at the time of inference, the learning model outputs the determination result of the presence or absence of drowsiness to the drowsiness presence-absence determination unit 15.

The drowsiness presence-absence determination unit 15 outputs an determination result of presence/absence of drowsiness to, for example, a driver monitoring system (not illustrated).

In the first embodiment described above, the drowsiness determination device 3 includes the image data acquiring unit 11 to acquire image data indicating a face image of a driver from the camera 1 that captures the face of the driver, the alertness level estimating unit 12 to estimate an alertness level indicating the degree of alertness of the driver on the basis of the image data acquired by the image data acquiring unit 11, the waveform information acquiring unit 13 to acquire waveform information indicating an electrocardiographic waveform of the driver from the sensor 2 that detects the electrocardiographic waveform of the driver, and the heartbeat feature amount calculating unit 14 to calculate a feature amount of a heartbeat of the driver from the electrocardiographic waveform indicated by the waveform information acquired by the waveform information acquiring unit 13. Further, the drowsiness determination device 3 includes the drowsiness presence-absence determination unit 15 to determine the presence or absence of drowsiness of the driver on the basis of the alertness level estimated by the alertness level estimating unit 12 and the feature amount of the heartbeat calculated by the heartbeat feature amount calculating unit 14. Therefore, the drowsiness determination device 3 can reduce false detection of drowsiness more than the device disclosed in Patent Literature 1.

In the drowsiness determination device 3 illustrated in FIG. 1, the alertness level estimation processing unit 12b gives the feature amount of the face after normalization to the learning model, and acquires the alertness level of the driver from the learning model. However, this is merely an example, and if the feature amount of the face after normalization is, for example, a proportion of the time during which the eyes are closed within a certain period, the alertness level estimation processing unit 12b may give a proportion of the time during which the eyes are closed within the certain period to a function that returns a larger alertness level as the proportion of the time during which the eyes are closed within the certain period is smaller, and acquire the alertness level of the driver from the function.

In addition, if the feature amount of the face after normalization is, for example, the number of blinks in a certain period, the alertness level estimation processing unit 12b may give the number of blinks in the certain period to a function that returns a larger alertness level as the number of blinks in the certain period is smaller, and acquire the alertness level of the driver from the function.

Furthermore, if the feature amount of the face after normalization is, for example, the number of yawns in a certain period, the alertness level estimation processing unit 12b may give the number of yawns in the certain period to a function that returns a larger alertness level as the number of yawns in the certain period is smaller, and acquire the alertness level of the driver from the function.

In the drowsiness determination device 3 illustrated in FIG. 1, the drowsiness presence-absence determination unit 15 gives the alertness level of the driver and the feature amount of the heartbeat after normalization to the learning model, and acquires the determination result of the presence or absence of drowsiness from the learning model. However, this is merely an example, and the drowsiness presence-absence determination unit 15 may determine that the driver is drowsy, for example, when the alertness level of the driver is smaller than a first determination threshold and the feature amount of the heartbeat after normalization is larger than a second determination threshold, and determine that the driver is not drowsy otherwise.

Each of the first determination threshold and the second determination threshold may be stored in an internal memory of the drowsiness presence-absence determination unit 15 or may be provided from the outside of the drowsiness determination device 3.

In addition, by accepting a change in each of the first determination threshold and the second determination threshold, the drowsiness presence-absence determination unit 15 may determine the presence or absence of drowsiness with emphasis on the alertness level of the driver rather than the feature amount of the heartbeat after normalization, or may determine the presence or absence of drowsiness with emphasis on the feature amount of the heartbeat after normalization rather than the alertness level of the driver.

In the drowsiness determination device 3 illustrated in FIG. 1, the facial feature amount calculating unit 12a outputs the feature amount of the face after normalization to the alertness level estimation processing unit 12b. When the driver in the vehicle is always the same person and there is no need to absorb the individual differences in the feature amounts of the face, the facial feature amount calculating unit 12a may output the feature amount of the face that is not normalized to the alertness level estimation processing unit 12b.

Further, in the drowsiness determination device 3 illustrated in FIG. 1, the heartbeat feature amount calculation processing unit 14b outputs the feature amount of the heartbeat after normalization to the drowsiness presence-absence determination unit 15. When the driver who gets into the vehicle is always the same person and it is not necessary to absorb the individual differences in the feature amounts of the heartbeat, the heartbeat feature amount calculation processing unit 14b may output the feature amount of the heartbeat that is not normalized to the drowsiness presence-absence determination unit 15.

### Second Embodiment

In a second embodiment, a drowsiness determination device 3 in which a heartbeat feature amount calculating unit 16 calculates a normalization coefficient of a heartbeat feature amount on the basis of a plurality of pieces of waveform information only when an alertness level estimated by an alertness level estimating unit 12 is equal to or more than a threshold will be described.

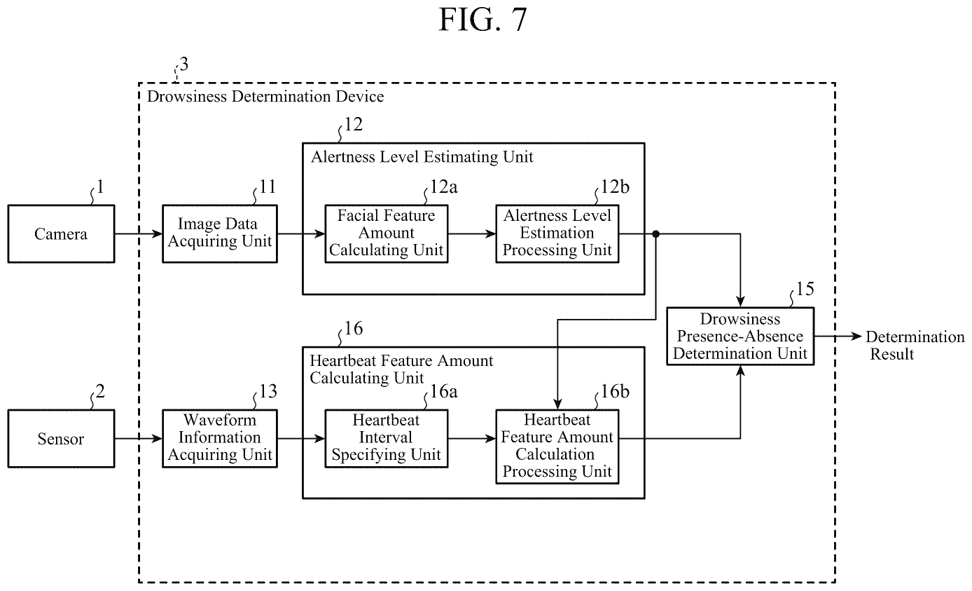

FIG. 7 is a configuration diagram illustrating the drowsiness determination device 3 according to the second embodiment. In FIG. 7, the same reference numerals as those in FIG. 1 denote the same or corresponding parts, and thus description thereof is omitted.

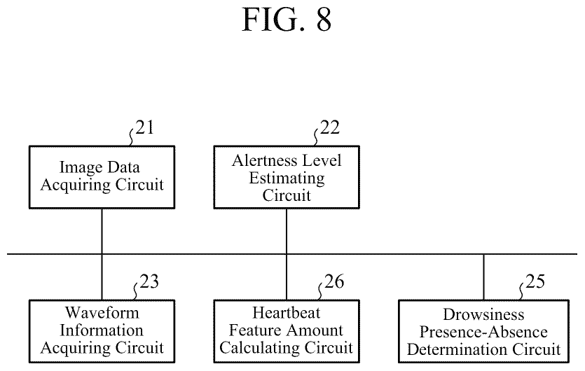

FIG. 8 is a hardware configuration diagram illustrating hardware of the drowsiness determination device 3 according to the second embodiment. In FIG. 8, the same reference numerals as those in FIG. 2 denote the same or corresponding parts, and thus description thereof is omitted.

The drowsiness determination device 3 illustrated in FIG. 7 includes an image data acquiring unit 11, the alertness level estimating unit 12, a waveform information acquiring unit 13, the heartbeat feature amount calculating unit 16, and a drowsiness presence-absence determination unit 15.

The heartbeat feature amount calculating unit 16 is implemented by, for example, a heartbeat feature amount calculating circuit 26 illustrated in FIG. 8.

The heartbeat feature amount calculating unit 16 includes a heartbeat interval specifying unit 16a and a heartbeat feature amount calculation processing unit 16b.

The heartbeat feature amount calculating unit 16 acquires waveform information from the waveform information acquiring unit 13, and acquires an alertness level of the driver from the alertness level estimating unit 12.

The heartbeat feature amount calculating unit 16 calculates the feature amount of the heartbeat of the driver from an electrocardiographic waveform indicated by the waveform information.

When the alertness level of the driver is equal to or more than a threshold, the heartbeat feature amount calculating unit 16 calculates a normalization coefficient of the feature amount of the heartbeat on the basis of the plurality of pieces of waveform information, and normalizes the feature amount of the heartbeat using the normalization coefficient.

When the alertness level of the driver is less than the threshold, the heartbeat feature amount calculating unit 16 normalizes the feature amount of the heartbeat using the normalization coefficient when the driver is not drowsy without calculating the normalization coefficient of the feature amount of the heartbeat.

The heartbeat feature amount calculating unit 16 outputs the feature amount of the heartbeat after normalization to the drowsiness presence-absence determination unit 15.

The heartbeat interval specifying unit 16a acquires the waveform information from the waveform information acquiring unit 13.

The heartbeat interval specifying unit 16a specifies a heartbeat interval of the driver on the basis of the electrocardiographic waveform indicated by the waveform information.

The heartbeat interval specifying unit 16a outputs the heartbeat interval of the driver to the heartbeat feature amount calculation processing unit 16b.

The heartbeat feature amount calculation processing unit 16b acquires the heartbeat interval of the driver from the heartbeat interval specifying unit 16a, and acquires the alertness level of the driver from the alertness level estimating unit 12.

The heartbeat feature amount calculation processing unit 16b calculates the feature amount of the heartbeat from the heartbeat interval.

When the alertness level of the driver is equal to or more than the threshold, the heartbeat feature amount calculation processing unit 16b calculates a normalization coefficient of the feature amount of the heartbeat on the basis of the plurality of pieces of waveform information acquired by the waveform information acquiring unit 13. The threshold may be stored in an internal memory of the heartbeat feature amount calculation processing unit 16b or may be given from the outside of the drowsiness determination device 3. The plurality of pieces of waveform information is a plurality of pieces of waveform information at different times of detection by the sensor 2.

The heartbeat feature amount calculation processing unit 16b normalizes the feature amount of the heartbeat using the normalization coefficient.

When the alertness level of the driver is less than the threshold, the heartbeat feature amount calculation processing unit 16b normalizes the feature amount of the heartbeat using the normalization coefficient when the driver is not drowsy without calculating the normalization coefficient of the feature amount of the heartbeat.

The heartbeat feature amount calculation processing unit 16b outputs the feature amount of the heartbeat after normalization to the drowsiness presence-absence determination unit 15.

In FIG. 7, it is assumed that each of the image data acquiring unit 11, the alertness level estimating unit 12, the waveform information acquiring unit 13, the heartbeat feature amount calculating unit 16, and the drowsiness presence-absence determination unit 15, which are components of the drowsiness determination device 3, is implemented by dedicated hardware as illustrated in FIG. 8. That is, it is assumed that the drowsiness determination device 3 is implemented by an image data acquiring circuit 21, an alertness level estimating circuit 22, a waveform information acquiring circuit 23, a heartbeat feature amount calculating circuit 26, and a drowsiness presence-absence determination circuit 25.

Further, each of the image data acquiring circuit 21, the alertness level estimating circuit 22, the waveform information acquiring circuit 23, the heartbeat feature amount calculating circuit 26, and the drowsiness presence-absence determination circuit 25 corresponds to, for example, a single circuit, a composite circuit, a programmed processor, a parallel-programmed processor, an application specific integrated circuit (ASIC), a field-programmable gate array (FPGA), or a combination thereof.

The components of the drowsiness determination device 3 are not limited to those implemented by dedicated hardware, and the drowsiness determination device 3 may be implemented by software, firmware, or a combination of software and firmware.

In a case where the drowsiness determination device 3 is implemented by software, firmware, or the like, a program for causing a computer to execute each processing procedure in the image data acquiring unit 11, the alertness level estimating unit 12, the waveform information acquiring unit 13, the heartbeat feature amount calculating unit 16, and the drowsiness presence-absence determination unit 15 is stored in the memory 31 illustrated in FIG. 3. Then, the processor 32 illustrated in FIG. 3 executes the program stored in the memory 31.

Further, FIG. 8 illustrates an example in which each of the components of the drowsiness determination device 3 is implemented by dedicated hardware, and FIG. 3 illustrates an example in which the drowsiness determination device 3 is implemented by software, firmware, or the like. However, this is merely an example, and some components in the drowsiness determination device 3 may be implemented by dedicated hardware, and the remaining components may be implemented by software, firmware, or the like.

Next, the operation of the drowsiness determination device 3 illustrated in FIG. 7 will be described. The drowsiness determination device 3 is similar to the drowsiness determination device 3 illustrated in FIG. 1 except for the heartbeat feature amount calculating unit 16. Thus, only the operation of the heartbeat feature amount calculating unit 16 will be described here.

The heartbeat interval specifying unit 16a acquires waveform information from the waveform information acquiring unit 13.

Similarly to the heartbeat interval specifying unit 14a illustrated in FIG. 1, the heartbeat interval specifying unit 16a specifies the heartbeat interval of the driver on the basis of the electrocardiographic waveform indicated by the waveform information.

The heartbeat interval specifying unit 16a outputs the heartbeat interval of the driver to the heartbeat feature amount calculation processing unit 16b.

The heartbeat feature amount calculation processing unit 16b acquires the heartbeat interval of the driver from the heartbeat interval specifying unit 16a, and acquires the alertness level of the driver from the alertness level estimating unit 12.

Similarly to the heartbeat feature amount calculation processing unit 14b illustrated in FIG. 1, the heartbeat feature amount calculation processing unit 16b calculates the feature amount of the heartbeat from the heartbeat interval.

The heartbeat feature amount calculation processing unit 16b acquires, for example, M pieces of waveform information including the latest waveform information and the past waveform information from the waveform information acquiring unit 13 via the heartbeat interval specifying unit 16a.

The heartbeat feature amount calculation processing unit 16b compares the alertness level of the driver with the threshold.

When the alertness level of the driver is equal to or more than the threshold, the heartbeat feature amount calculation processing unit 16b calculates a normalization coefficient of the feature amount of the heartbeat on the basis of the M pieces of waveform information, similarly to the heartbeat feature amount calculation processing unit 14b illustrated in FIG. 1.

Similarly to the heartbeat feature amount calculation processing unit 14b illustrated in FIG. 1, the heartbeat feature amount calculation processing unit 16b normalizes the feature amount of the heartbeat using the normalization coefficient.

When the alertness level of the driver is less than the threshold, the heartbeat feature amount calculation processing unit 16b normalizes the feature amount of the heartbeat using the normalization coefficient when the driver is not drowsy without calculating the normalization coefficient of the feature amount of the heartbeat. The normalization coefficient when the driver is not drowsy may be stored in the internal memory of the heartbeat feature amount calculation processing unit 16b or may be given from the outside of the drowsiness determination device 3.

Furthermore, the normalization coefficient when the driver is not drowsy may be, for example, a normalization coefficient calculated by the heartbeat feature amount calculation processing unit 16b until a certain time elapses from the time point when the driver gets into the vehicle.

In a case where the normalization coefficient of the feature amount of the heartbeat is calculated on the basis of the waveform information when the driver is drowsy, the feature amount of the heartbeat after normalization does not reflect the intensity of drowsiness much. As a result, even if the driver is drowsy, the determination result indicating that the driver is drowsy may not be obtained from the drowsiness presence-absence determination unit 15. Therefore, the heartbeat feature amount calculation processing unit 16b normalizes the feature amount of the heartbeat using the normalization coefficient when the driver is not drowsy.

The heartbeat feature amount calculation processing unit 16b outputs the feature amount of the heartbeat after normalization to the drowsiness presence-absence determination unit 15.

In the second embodiment described above, when the alertness level estimated by the alertness level estimating unit 12 is equal to or more than the threshold, the heartbeat feature amount calculating unit 16 calculates a normalization coefficient of the feature amount of the heartbeat on the basis of the plurality of pieces of waveform information, and normalizes the feature amount of the heartbeat using the normalization coefficient. The drowsiness determination device 3 illustrated in FIG. 7 is configured in such a manner that when the alertness level estimated by the alertness level estimating unit 12 is less than the threshold, the heartbeat feature amount calculating unit 16 normalizes the feature amount of the heartbeat using the normalization coefficient when the driver is not drowsy without calculating the normalization coefficient of the feature amount of the heartbeat. Therefore, the drowsiness determination device 3 illustrated in FIG. 7 can enhance the drowsiness sensing accuracy more than drowsiness determination device 3 illustrated in FIG. 1.

### Third Embodiment

In a third embodiment, a drowsiness determination device 3 will be described in which, if an alertness level estimated by an alertness level estimating unit 12 is less than a threshold, a drowsiness presence-absence determination unit 17 determines the presence or absence of drowsiness of the driver on the basis of the alertness level.

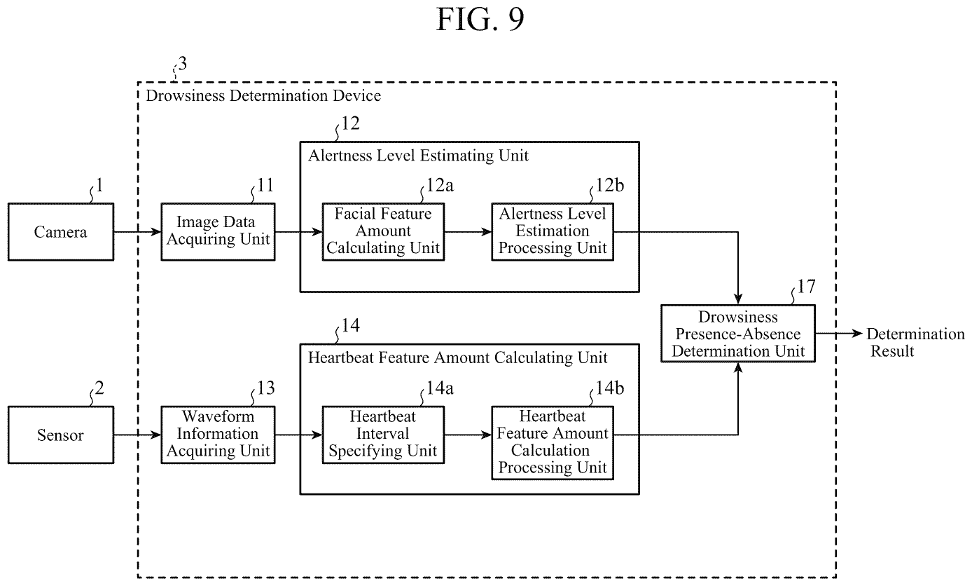

FIG. 9 is a configuration diagram illustrating the drowsiness determination device 3 according to the third embodiment. In FIG. 9, the same reference numerals as those in FIGS. 1 and 7 denote the same or corresponding parts, and thus description thereof is omitted.

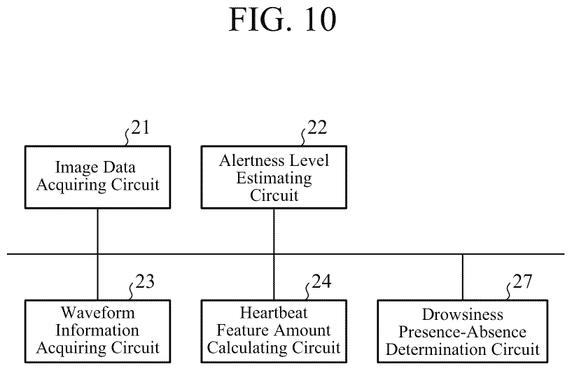

FIG. 10 is a hardware configuration diagram illustrating hardware of the drowsiness determination device 3 according to the third embodiment. In FIG. 10, the same reference numerals as those in FIGS. 2 and 8 denote the same or corresponding parts, and thus description thereof is omitted.

The drowsiness determination device 3 illustrated in FIG. 9 includes an image data acquiring unit 11, an alertness level estimating unit 12, a waveform information acquiring unit 13, a heartbeat feature amount calculating unit 14, and a drowsiness presence-absence determination unit 17.

The drowsiness presence-absence determination unit 17 is implemented by, for example, a drowsiness presence-absence determination circuit 27 illustrated in FIG. 10.

The drowsiness presence-absence determination unit 17 acquires an alertness level of a driver from an alertness level estimation processing unit 12b, and acquires a feature amount of the heartbeat after normalization from a heartbeat feature amount calculation processing unit 14b.

When the alertness level of the driver is equal to or more than the threshold, the drowsiness presence-absence determination unit 17 determines the presence or absence of drowsiness of the driver on the basis of the alertness level of the driver and the feature amount of the heartbeat after normalization, similarly to the drowsiness presence-absence determination unit 15 illustrated in FIG. 1. The threshold may be stored in an internal memory of the drowsiness presence-absence determination unit 17 or may be given from the outside of the drowsiness determination device 3.

When the alertness level of the driver is less than the threshold, the drowsiness presence-absence determination unit 17 determines the presence or absence of drowsiness of the driver on the basis of the alertness level of the driver.

In FIG. 9, it is assumed that each of the image data acquiring unit 11, the alertness level estimating unit 12, the waveform information acquiring unit 13, the heartbeat feature amount calculating unit 14, and the drowsiness presence-absence determination unit 17, which are components of the drowsiness determination device 3, is implemented by dedicated hardware as illustrated in FIG. 10. That is, it is assumed that the drowsiness determination device 3 is implemented by an image data acquiring circuit 21, an alertness level estimating circuit 22, a waveform information acquiring circuit 23, a heartbeat feature amount calculating circuit 24, and a drowsiness presence-absence determination circuit 27.

Further, each of the image data acquiring circuit 21, the alertness level estimating circuit 22, the waveform information acquiring circuit 23, the heartbeat feature amount calculating circuit 24, and the drowsiness presence-absence determination circuit 27 corresponds to, for example, a single circuit, a composite circuit, a programmed processor, a parallel-programmed processor, an application specific integrated circuit (ASIC), a field-programmable gate array (FPGA), or a combination thereof.

The components of the drowsiness determination device 3 are not limited to those implemented by dedicated hardware, and the drowsiness determination device 3 may be implemented by software, firmware, or a combination of software and firmware.

In a case where the drowsiness determination device 3 is implemented by software, firmware, or the like, a program for causing a computer to execute each processing procedure in the image data acquiring unit 11, the alertness level estimating unit 12, the waveform information acquiring unit 13, the heartbeat feature amount calculating unit 14, and the drowsiness presence-absence determination unit 17 is stored in the memory 31 illustrated in FIG. 3. Then, the processor 32 illustrated in FIG. 3 executes the program stored in the memory 31.

Further, FIG. 10 illustrates an example in which each of the components of the drowsiness determination device 3 is implemented by dedicated hardware, and FIG. 3 illustrates an example in which the drowsiness determination device 3 is implemented by software, firmware, or the like. However, this is merely an example, and some components in the drowsiness determination device 3 may be implemented by dedicated hardware, and the remaining components may be implemented by software, firmware, or the like.

Next, the operation of the drowsiness determination device 3 illustrated in FIG. 9 will be described. The drowsiness determination device 3 is similar to the drowsiness determination device 3 illustrated in FIG. 1 except for the drowsiness presence-absence determination unit 17. Thus, only the operation of the drowsiness presence-absence determination unit 17 will be described here.

The drowsiness presence-absence determination unit 17 acquires the alertness level of the driver from the alertness level estimation processing unit 12b, and acquires the feature amount of the heartbeat after normalization from the heartbeat feature amount calculation processing unit 14b.

The drowsiness presence-absence determination unit 17 compares the alertness level of the driver with a threshold.

When the alertness level of the driver is equal to or more than the threshold, the drowsiness presence-absence determination unit 17 determines the presence or absence of drowsiness of the driver on the basis of the alertness level of the driver and the feature amount of the heartbeat after normalization, similarly to the drowsiness presence-absence determination unit 15 illustrated in FIG. 1.

When the alertness level of the driver is less than the threshold, the drowsiness presence-absence determination unit 17 determines the presence or absence of drowsiness of the driver on the basis of the alertness level of the driver.

Specifically, if the alertness level of the driver is equal to or more than a set value, the drowsiness presence-absence determination unit 17 determines that the driver is drowsy.

If the alertness level of the driver is less than the set value, the drowsiness presence-absence determination unit 17 determines that the driver is not drowsy. The set value may be stored in an internal memory of the drowsiness presence-absence determination unit 17 or may be given from the outside of the drowsiness determination device 3.

The drowsiness presence-absence determination unit 17 outputs an determination result of presence/absence of drowsiness to, for example, a driver monitoring system (not illustrated).

In the third embodiment described above, if the alertness level estimated by the alertness level estimating unit 12 is equal to or more than the threshold, the drowsiness presence-absence determination unit 17 determines the presence or absence of drowsiness of the driver on the basis of the alertness level and the feature amount of the heartbeat after normalization output from the heartbeat feature amount calculating unit 14. The drowsiness determination device 3 illustrated in FIG. 9 is configured in such a manner that, when the alertness level estimated by the alertness level estimating unit 12 is less than the threshold, the drowsiness presence-absence determination unit 17 determines the presence or absence of drowsiness of the driver on the basis of the alertness level. Therefore, the drowsiness determination device 3 illustrated in FIG. 9 can improve drowsiness sensing accuracy more than the drowsiness determination device 3 illustrated in FIG. 1.

Note that, in the present disclosure, free combinations of the embodiments, modifications of any components of the embodiments, or omissions of any components in the embodiments are possible.

### INDUSTRIAL APPLICABILITY

The present disclosure is suitable for a drowsiness determination device and a drowsiness determination method.

### REFERENCE SIGNS LIST

1: camera, 2: sensor, 3: drowsiness determination device, 11: image data acquiring unit, 12: alertness level estimating unit, 12a: facial feature amount calculating unit, 12b: alertness level estimation processing unit, 13: waveform information acquiring unit, 14: heartbeat feature amount calculating unit, 14a: heartbeat interval specifying unit, 14b: heartbeat feature amount calculation processing unit, 15: drowsiness presence-absence determination unit, 16: heartbeat feature amount calculating unit, 16a: heartbeat interval specifying unit, 16b: heartbeat feature amount calculation processing unit, 17: drowsiness presence-absence determination unit, 21: image data acquiring circuit, 22: alertness level estimating circuit, 23: waveform information acquiring circuit, 24: heartbeat feature amount calculating circuit, 25: drowsiness presence-absence determination circuit, 26: heartbeat feature amount calculating circuit, 27: drowsiness presence-absence determination circuit, 31: memory, 32: processor

1.-10. (canceled)

11. A drowsiness determination device comprising:
processing circuitry configured to
acquire image data indicating a face image of a driver from a camera that captures a face of the driver;
estimate an alertness level indicating a degree of alertness of the driver on a basis of the acquired image data;
acquire waveform information indicating an electrocardiographic waveform of the driver from a sensor that detects an electrocardiographic waveform of the driver;
calculate a feature amount of a heartbeat of the driver from the electrocardiographic waveform indicated by the acquired waveform information;
determine presence or absence of drowsiness of the driver on a basis of the estimated alertness level and the calculated feature amount of the heartbeat;
acquire a plurality of pieces of waveform information at different times of detection by the sensor;
calculate a normalization coefficient of the feature amount of the heartbeat on a basis of the plurality of pieces of waveform information, normalize the feature amount of the heartbeat using the normalization coefficient, and output the feature amount of the heartbeat after normalization; and
determine the presence or absence of drowsiness of the driver on a basis of the estimated alertness level and the outputted feature amount of the heartbeat after normalization.

12. The drowsiness determination device according to claim 11, wherein
the processing circuitry is further configured to calculate a feature amount of the face of the driver from the acquired image data and estimate the alertness level of the driver on a basis of the feature amount of the face.

13. The drowsiness determination device according to claim 12, wherein
the processing circuitry is further configured to acquire a plurality of pieces of image data at different times of capturing by the camera and calculate a normalization coefficient of the feature amount of the face on a basis of the plurality of pieces of image data, normalize the feature amount of the face using the normalization coefficient, and estimate the alertness level of the driver on a basis of the feature amount of the face after normalization.

14. The drowsiness determination device according to claim 11, wherein
the processing circuitry is further configured to specify a heartbeat interval of the driver on a basis of the electrocardiographic waveform indicated by the acquired waveform information and calculate the feature amount of the heartbeat from the heartbeat interval.

15. The drowsiness determination device according to claim 11, wherein
if the estimated alertness level is equal to or more than a threshold,
the processing circuitry is further configured to calculate a normalization coefficient of the feature amount of the heartbeat on a basis of the plurality of pieces of waveform information, normalize the feature amount of the heartbeat using the normalization coefficient, and if the estimated alertness level is less than the threshold, the processing circuitry is further configured to normalize the feature amount of the heartbeat using a normalization coefficient when the driver is not drowsy without calculating the normalization coefficient of the feature amount of the heartbeat.

16. The drowsiness determination device according to claim 11, wherein
if the estimated alertness level is equal to or more than a threshold,
the processing circuitry is further configured to determine the presence or absence of drowsiness of the driver on a basis of the alertness level and the feature amount of the heartbeat after normalization having been outputted, and if the alertness level is less than the threshold, the processing circuitry is further configured to determine the presence or absence of drowsiness of the driver on a basis of the alertness level.

17. The drowsiness determination device according to claim 11, wherein
the processing circuitry is further configured to
give the estimated alertness level and the calculated feature amount of the heartbeat to a learning model in which the presence or absence of drowsiness is learned by being given the alertness level of the driver, the feature amount of the heartbeat of the driver, and teacher data indicating the presence or absence of drowsiness at the time of learning, and acquire a determination result of the presence or absence of drowsiness of the driver from the learning model.

18. The drowsiness determination device according to claim 11, wherein
based on the electrocardiographic waveform indicated by the acquired waveform information,
the processing circuitry is further configured to calculate, as the feature amount of the heartbeat of the driver, an average value of the heartbeat interval of the driver within a certain period, a standard deviation within a certain period of the heartbeat interval of the driver, an average value within a certain period of the heartbeat of the driver, a square root of an average value within a certain period of a square of a difference between two heartbeat intervals adjacent in a time direction, a number of times indicating that a difference between two heartbeat intervals adjacent in the time direction becomes larger than a threshold within a certain period, or a ratio at which a difference between two heartbeat intervals adjacent in the time direction becomes larger than a threshold within a certain period.

19. A drowsiness determination method comprising:
acquiring image data indicating a face image of a driver from a camera that captures a face of the driver;
estimating an alertness level indicating a degree of alertness of the driver on a basis of the acquired image data;
acquiring waveform information indicating an electrocardiographic waveform of the driver from a sensor that detects an electrocardiographic waveform of the driver;
calculating a feature amount of a heartbeat of the driver from the electrocardiographic waveform indicated by the acquired waveform information;
determining presence or absence of drowsiness of the driver on a basis of the estimated alertness level and the calculated feature amount of the heartbeat,
acquiring a plurality of pieces of waveform information at different times of detection by the sensor;
calculating a normalization coefficient of the feature amount of the heartbeat on a basis of the plurality of pieces of waveform information, normalizing the feature amount of the heartbeat using the normalization coefficient, and outputting the feature amount of the heartbeat after normalization; and
determining the presence or absence of drowsiness of the driver on a basis of the estimated alertness level and the feature amount of the heartbeat after normalization having been output.
